(19) United States
(12) Patent Application Publication
(10) Pub. No.: US 2020/0383807 A1
(43) Pub. Date: Dec. 10, 2020

(54) PROSTHESIS SOCKET SYSTEM

(71) Applicant: Ottobock SE & Co. KGaA, Duderstadt (DE)

(72) Inventors: Jens VOLKMAR, Duderstadt (DE); Harald GOTTLIEB, Jützenbach (DE); Andreas LEINIGER, Leinefelde (DE); Sebastian BULS, Duderstadt (DE)

(73) Assignee: Ottobock SE & Co. KGaA, Duderstadt (DE)

(21) Appl. No.: 15/733,225

(22) PCT Filed: Dec. 13, 2018
(86) PCT No.: PCT/EP2018/084726

§ 371 Date: Jun. 11, 2020

(30) Foreign Application Priority Data
Dec. 14, 2017 (DE) 10 2017 129 930.0

(51) Int. Cl. A61F 2/80; A61F 2/78; A61F 2/60

(52) U.S. Cl. CPC A61F 2/80; A61F 2002/5007; A61F 2/60; A61F 2/7812

(57) ABSTRACT

A prosthesis socket system with a rigid outer socket which has a proximal outer socket opening for receiving an extremity stump of a wearer of the prosthesis socket system, and a flexible inner socket, which is connected to the outer socket and has a proximal inner socket opening for receiving the extremity stump, wherein at least one sealing lip is arranged on an inner side of the inner socket facing the extremity stump.

The invention relates to a prosthesis socket system with a rigid outer socket, which has a proximal outer socket opening for receiving an extremity stump of a wearer of the prosthesis socket system, and with a flexible inner socket, which is connected to the outer socket and has a proximal inner socket opening for receiving the extremity stump.

A prosthesis socket system of this kind is known, for example, from DE 10 2011 117 801 A1. Prosthesis socket systems of this kind are often configured as leg prostheses, for example for patients who haver undergone transtibial or transfemoral amputation. To ensure that the prosthesis socket system can be fitted firmly and securely to the extremity stump of the wearer, a negative pressure is usually generated between the inner socket and the extremity stump. The advantage of prosthesis socket systems of this kind, compared to non-flexible prosthesis sockets, is that fluctuations in the volume of the extremity stump can be brought under control, since adaptation of the flexible inner socket to the extremity stump takes place. However, on account of the flexible inner socket, other challenges are posed when applying the prosthesis socket system to the extremity stump. It is desirable to achieve a firm, secure and comfortable hold by simple design means.

The problem addressed by the invention is therefore to make available a prosthesis socket system which provides a secure hold of the extremity stump of a patient in the inner socket of the prosthesis socket system by simple design means and at the same time with a high level of comfort for the wearer.

The invention solves the stated problem by providing a prosthesis socket system having the features of the main claim. Advantageous embodiments and developments of the invention are disclosed in the dependent claims, the description and the figures.

In the prosthesis socket system with a rigid outer socket, which has a proximal outer socket opening for receiving an extremity stump of a wearer of the prosthesis socket system, and with a flexible inner socket, which is connected to the outer socket and has a proximal inner socket opening for receiving the extremity stump, provision is made that at least one sealing lip is arranged on an inner face of the inner socket directed toward the extremity stump.

A space between the inner socket and the extremity stump is sealed off by the sealing lip. If a negative pressure is established between the extremity stump and the inner socket, the sealing lip serves to seal off the interior between inner socket and extremity stump from the environment. The sealing lip is drawn in the direction of the negative pressure and places itself on the extremity stump. It was hitherto customary for a sealing element, for example a knee sleeve, to be pulled over the prosthesis socket system and the extremity stump in order to better seal the inner socket and the outer socket from the environment and to provide a better hold of the inner socket on the extremity stump by means of negative pressure. With the prosthesis socket system according to the invention, secure fastening of the prosthesis socket system to the patient is still possible, but without the need for a cuff engaging round the outer socket, since the sealing lip itself can already provide sufficient fixing by negative pressure. The prosthesis socket system according to the invention can therefore be fitted in place particularly simply and quickly. It can be worn with or without a prosthesis liner. With the prosthesis socket system according to the invention, oval and also irregularly shaped socket cross sections can be sealed off. It is equally suitable for transcarpal and transradial stumps.

The rigid outer socket is preferably made largely or completely from a rigid material, for example a fiber-reinforced plastic, in particular a carbon-fiber-reinforced or glass-fiber-reinforced plastic. These materials are easy to process and are stable, and yet they have a very low inherent weight. In this way, it is possible to produce a particularly light prosthesis socket that has good stability properties. Particularly on account of the high degree of inherent stability of the outer socket, the person wearing the prosthesis socket system according to the invention is afforded a feeling of enhanced safety. The high degree of stability and strength of the outer socket also permits a secure and stable arrangement of distal prosthetic devices, for example a prosthetic foot, or a prosthetic knee joint with lower-leg tube and prosthetic foot.

The flexible inner socket is preferably made largely or completely from a flexible material. The flexible material preferably has good cushioning properties and elastic properties. As a flexible material for the inner socket, a silicone is preferably used, for example a HTV silicone, which has a high breathing activity and does not limit the muscle functions of the extremity stump. The elasticity of the silicone can be limited, according to requirements, by the incorporation of a woven fabric. In addition or alternatively, the flexible inner socket can have a padding on its inner face. The padding can be arranged substantially on the whole surface or part of the surface of the inner face of the inner socket, if appropriate with the exception of regions around the sealing lip. Alternatively or in addition, the padding can be arranged on defined zones of the inner face of the inner socket. For example, padding can be provided only in zones, or additionally in zones, in which particularly high pressure occurs when the inner socket is placed onto the extremity stump.

The connection of the inner socket to the outer socket is preferably a connection produced mainly by form-fit and/or force-fit engagement, preferably a releasable connection, for example a screw connection or a connection by means of hook-and-loop fasteners or interlocking elements. In addition or alternatively, a cohesively bonded connection of the inner socket to the outer socket is also possible.

According to the invention, at least one sealing lip is arranged on an inner face of the inner socket directed toward the extremity stump. However, it is also possible for two, three, four or more sealing lips to be arranged on the inner face of the inner socket. Several sealing lips can be arranged directly alongside each other and/or spaced apart from each other in the direction of a proximal-distal extent of the inner socket and/or spaced apart from each other along an inner circumference of the inner socket.

In a development of the invention, the sealing lip is configured as a peripheral sealing lip, which is arranged along a complete inner circumference of the inner face of the inner socket. This has the advantage that complete sealing can take place with just one sealing lip along the entire inner circumference of the inner socket and thus the entire circumference of the extremity stump. It is also possible for several peripheral sealing lips to be arranged spaced apart from each other along the proximal-distal extent of the inner socket. This can provide additional hold, for example if the extremity stump is irregular in shape in the direction of its proximal-distal extent. For example, if the extremity stump is not fully enclosed along its circumference by a first peripheral sealing lip, such that the required negative pressure for securely holding the inner socket cannot be produced, the negative pressure for a secure hold can be generated by a second or third peripheral sealing lip or further peripheral sealing lips which can be arranged distally or proximally from the first peripheral sealing lip, depending on the direction or directions in which a negative pressure acts. It is additionally possible to provide one or more interruptions in the form of slits or material recesses in the sealing lip along the profile of the peripheral sealing lip, in order, by means of another pressure distribution, to relieve the forces acting on the extremity stump.

According to a development of the invention, at least one window is formed in the outer socket, in particular for relieving a pressure applied to the extremity stump. Such a window reduces the weight of the outer socket and gives the wearer of the prosthesis socket system the ability to notice environmental influences in this region. In particular, at least one window is formed, although preferably two, three, four or more windows are formed in the outer socket. The windows are advantageously adapted to the anatomy of the extremity or of the extremity stump to be treated, e.g. to bone or musculature. The at least one window is preferably filled at least partially, in particular completely, by the flexible inner socket. For this purpose, the flexible inner socket preferably has a completely closed design, except for its proximal inner socket opening and possible recesses, for example for connection to the outer socket or for receiving a release valve or a suction attachment. In particular, it is preferable if the inner socket and the outer socket are each closed in a distal region by a respective cap.

In a development of the invention, the outer socket is configured as a lower-leg socket, wherein a window can be arranged in a frontal shin region and/or a distal calf region and/or in the region of a head of a fibula. In an embodiment of the outer socket as a lower-leg socket, a problem that can arise is that bony structures press against the prosthesis socket system. Even if the inner socket is made of a comparatively soft, flexible and preferably elastic material, the rigid outer socket creates contact points which can lead to pain in the region of the bony structures. To permit pressure relief in these regions, provision is made that a window is provided in the regions with exposed bony structures. Thus, for example, a window can be arranged in a frontal shin region, a distal calf region and/or in the region of the head of a fibula, in order to permit local pressure relief in said regions. A corresponding window can likewise be formed in both regions. The prosthesis socket system can also be configured to receive a thigh stump or a stump of the upper extremity. In the case of a thigh socket, the window or windows serve as a possibility of coping with volume fluctuations; bony structures are not to be expected at the circumference of the outer socket. In a prosthesis socket system for the upper extremity, windows can be arranged in the region of prominent bony structures, for example in the elbow region.

Furthermore, a window can be arranged in a calf region of the outer socket in order to permit pressure relief in this region too.

In a development of the invention, provision is made that an adjustable insert, which is fastened to the outer socket, is arranged in at least one of the windows. Such an insert can take up the contour of the outer socket and substantially follow the contour of the window. A padding can be arranged on the inner face of the insert, i.e. on the face directed toward the inner socket, such that, when the insert is arranged in the window and suitably fastened, for example via a strap, a cable system, buckles or the like, the insert substantially supplements the outer contour of the outer socket and additionally applies pressure to the inner socket. With inserts inserted in windows, a padding is in particular provided in those regions that have no bony structures, for example, in the case of a thigh socket, at almost any desired location, since soft tissue almost completely surrounds the femur. In the case of a lower-leg socket, it would be expedient to provide padding at the insert in the region of the calf muscles, so as to be able to compensate for fluctuations in volume and, for example when the stump swells over the course of a day, to permit pressure relief in the soft tissue regions, or, when the swelling of the stump subsides and a clamping device is tightened, so as to be able to exert an increased pressure on the inner socket and therefore on the soft tissue. In this way, an enhanced feeling of safety can be afforded to the patient.

According to a development of the invention, the sealing lip is at least partially arranged in a region of the inner socket that lies inside the window, i.e. in which the inner socket is not covered by the material of the outer socket. Since the sealing lip has a high degree of flexibility in the region of a window, the sealing lip can rest more easily and more comfortably on the extremity stump, without a high pressure being exerted on the stump. In order to achieve an enhanced sealing action, the sealing lip is arranged in those regions of the inner socket that are covered by material of the outer socket, such that the inner socket with the sealing lip cannot escape outward.

In a development of the invention, the sealing lip is arranged closer to the proximal inner socket opening than to a distal end of the inner socket. In one embodiment of the invention, provision is made that the sealing lip is arranged in a proximal portion of the inner socket at a position which, measured from the proximal inner socket opening toward a distal end of the inner socket, amounts to one third or one quarter of the length of the inner socket from the proximal inner socket opening to the distal end of the inner socket. This has the advantage that a large surface area is made available for contact and thus adherence of the extremity stump to the inner socket. A particularly secure hold can be achieved in this way.

In a development of the invention, the sealing lip is arranged in one plane and thus has a rectilinear profile, which can also be inclined with respect to a chosen reference direction, for example the direction of gravity in the embodiment of the prosthesis socket, i.e. is not orthogonal to the direction of gravity. This plane can, for example, run transversely, in particular orthogonally, with respect to a proximal-distal extent of the inner socket. The extremity stump is in this way sealed off at its circumference along one plane.

In a development of the invention, the sealing lip is at least partially parallel to a contour of an inner socket edge surrounding the inner socket opening or to a contour of an outer socket edge surrounding the outer socket opening. This is particularly preferred for a contour of the inner socket edge or outer socket edge in the form of a three-dimensional curve. An example of a three-dimensional curve can have a bottom in the ventral region and dorsal region and a peak in the lateral region and medial region. In the proximal region of the prosthesis socket system, a contour of this kind has the effect that relief is obtained in the ventral region and dorsal region and strengthening is obtained in the medial region and lateral region. By virtue of the configuration of the sealing lip parallel to this contour, the distance from the sealing lip to the contour is advantageously kept constant. Such a sealing lip can also better compensate for irregularities of the stump volume and can improve the wearing comfort.

In a development of the invention, the sealing lip is cohesively bonded to the inner socket or formed integrally thereon, and/or the sealing lip and the inner socket are made of the same material. This permits simple manufacture and a high degree of stability.

Alternatively, the sealing lip is fastened to the inner socket by form-fit engagement and in particular exchangeably, in particular by being placed in a peripheral groove.

Advantageously, the material thicknesses of the sealing lip and of the inner socket differ. A material thickness of the sealing lip is preferably 5% to 40%, particularly preferably 10% to 30%, of the material thickness of the inner socket. Greater flexibility of the sealing lip compared to the inner socket is achieved in this way, such that the sealing lip can easily rest on the extremity stump.

In a development of the invention, the prosthesis socket system has a release valve and/or a suction attachment for regulating a negative pressure between the extremity stump and the inner socket. For this purpose, recesses are preferably formed in the inner socket and outer socket, into which recesses the release valve and/or the suction attachment are inserted. If the prosthesis socket system according to the invention is designed for a lower extremity, the interior between inner socket and extremity stump can be evacuated during walking, since the air located in the interior is expelled by the amputation stump when the foot of the prosthetic leg is placed onto the ground. A manual or electric pump, by means of which the interior can already be evacuated directly after placement into the before the use of the prosthesis, can be attached to a suction attachment. During the use of the prosthesis, the interior can additionally be evacuated by a pump. The prosthesis socket system according to the invention can have only a single release valve and/or suction attachment. However, it is also possible for several release valves and/or suction attachments to be used in the prosthesis socket system.

In a development of the invention, the inner socket and the outer socket are connected to each other releasably and in a manner free of pistoning, in particular by a screw connection with locking nuts integrated in the inner socket. Free of pistoning means that, during the movement of the extremity stump fitted with the prosthesis socket system, the inner socket and the outer socket are moved with each other, and without movement, or without any appreciable movement, relative to each other.

In a development of the invention, the outer socket edge is made, in a ventral region, from a flexible and non-elastic material, preferably from a fiber-reinforced plastic, in particular from a high-strength carbon fiber, aramid fiber or polyethylene fiber, wherein the flexible and non-elastic material is adapted to the shape of an extremity stump of a person wearing the prosthesis socket. This does not necessarily signify an individual adaptation to each particular prosthesis wearer, but also a standardized adaptation to the expected shape of the extremity stump of a prosthesis wearer.

The inner socket can be elastic at least in some regions or can be made of an elastic material at least in some regions. In this way, it is possible to compensate for changes in volume or to reduce pressure peaks.

Illustrative embodiments of the invention are explained below with reference to the accompanying figures, in which:

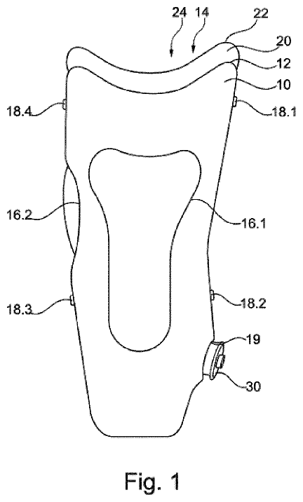

FIG. 1 shows a frontal view of an illustrative embodiment of a prosthesis socket system according to the invention,

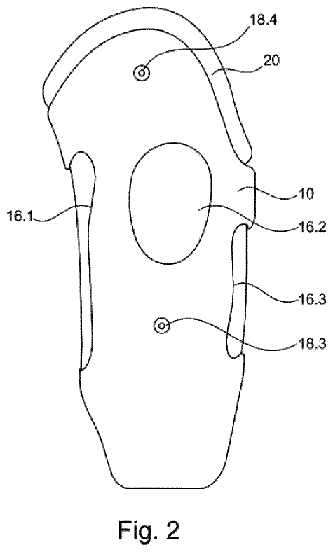

FIG. 2 shows a lateral view of an illustrative embodiment of a prosthesis socket system according to the invention,

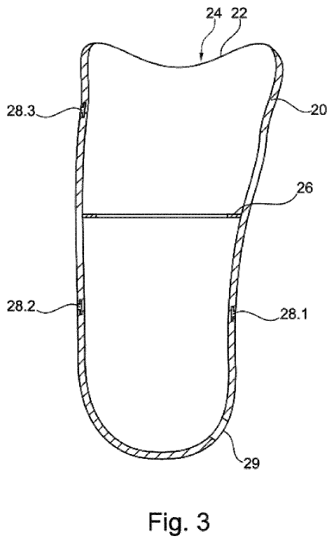

FIG. 3 shows a frontal cross-sectional view of an inner socket of an illustrative embodiment of the prosthesis socket system according to the invention,

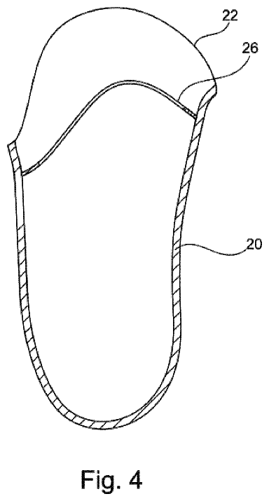

FIG. 4 shows a medial cross-sectional view of an inner socket of an illustrative embodiment of the prosthesis socket system according to the invention,

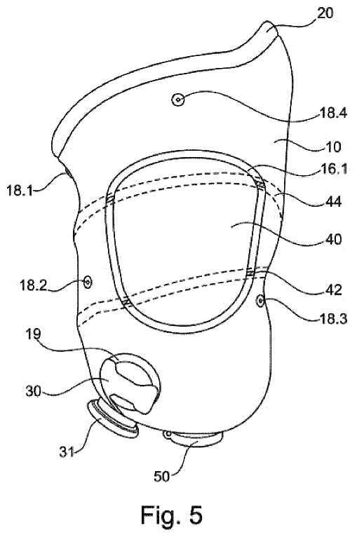

FIG. 5 shows a frontal view of a thigh socket,

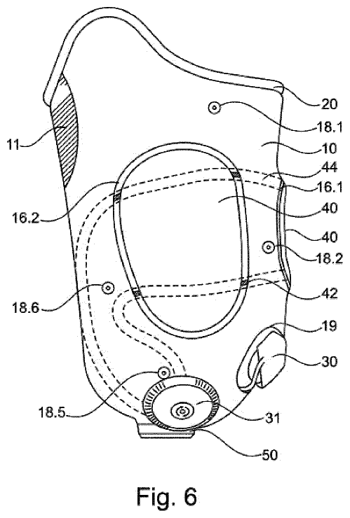

FIG. 6 shows an approximated lateral-medial view of a thigh socket according to FIG. 5,

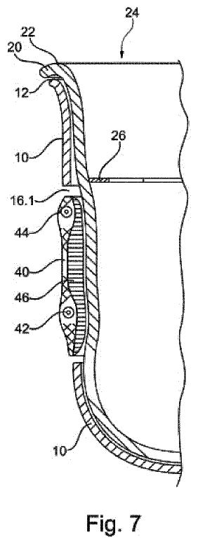

FIG. 7 shows a partial sectional view of a prosthesis socket system,

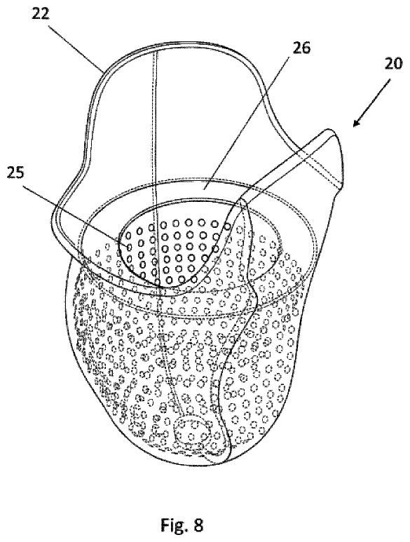

FIG. 8 shows a perspective view of a variant of an inner socket, and

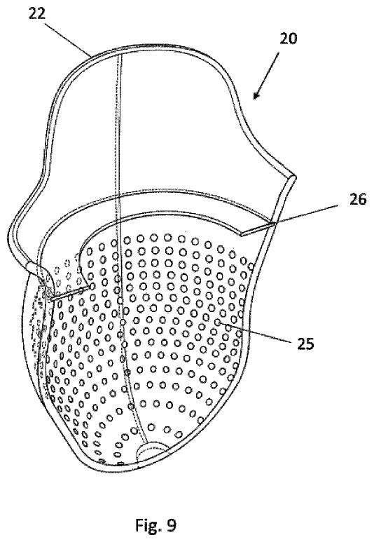

FIG. 9 shows a sectional view according to FIG. 8.

FIG. 1 shows an illustrative embodiment of a prosthesis socket system according to the invention in a frontal view. The prosthesis socket system in question here is a prosthesis socket system for a right lower leg of a patient who has undergone transtibial amputation. The prosthesis socket system has a rigid outer socket 10 and a flexible inner socket 20. The flexible inner socket 20 is arranged in the rigid outer socket 10, wherein the inner socket 20 protrudes from the outer socket 10 in the proximal direction. In the direction of a proximal outer socket opening 14, the outer socket 10 has an outer socket edge 12. In the direction of a proximal inner socket opening 24, the inner socket 20 has an inner socket edge 22. The outer socket opening 14 and the inner socket opening 24 serve to receive the right lower-leg stump of the patient.

Two windows 16.1 and 16.2 are formed in the outer socket 10 and are filled by the inner socket 20. An outer face of the inner socket 20 is to this extent visible in the windows 16.1 and 16.2. The frontal window 16.1 is designed in shape, arrangement and size to relieve the patient's tibia. The lateral window 16.2 is designed in shape, arrangement and size to relieve the head of the fibula of the patient. The shape, arrangement and size of the windows can be individually adapted to the anatomy for example of the particular prosthesis wearer or of an average person or to different body sizes or sexes. The outer socket 10 is connected to the inner socket 20 by means of screws 18.1, 18.2, 18.3 and 18.4. The screws 18.1 and 18.2 lie on a medial side of the prosthesis socket system, and the screws 18.3 and 18.4 lie on a lateral side of the prosthesis socket system. The number and arrangement of the screws 18.1 to 18.4 are shown here purely by way of example. It is also possible to provide more or fewer screws and/or to choose other positions. In particular, for connecting the outer socket 10 to the inner socket 20, a connection technique other than screwing can be chosen, for example a releasable form-fit connection via clips, hook-and-loop fasteners, interlocking regions, or other reversible connection systems.

An outer socket recess 19 for receiving a release valve is arranged medially in the outer socket 10. Alternatively or in addition, a suction attachment can be arranged in the outer socket recess 19, or at least one further outer socket recess 19 can be formed in the outer socket 10, in order to insert a second release valve or to attach a pump.

FIG. 2 shows a lateral view of the prosthesis socket system from FIG. 1. The lateral window 16.2 for relieving the head of the fibula can be seen particularly clearly here. The dorsal arrangement of the window 16.3 for relieving the distal calf region can also be seen. The lateral screws 18.3 and 18.4, which connect the outer socket 10 and the inner socket 20 to each other on this side of the prosthesis socket system, can be clearly seen. A further window 16.1 is present on the ventral or frontal face of the outer socket 10 and relieves a region of the tibia. The inner socket 20 also fills all of the windows 16.1, 16.2 and 16.3 here, such that the outer face of the inner socket 20 is visible in each of the windows 16.1, 16.2 and 16.3. The contours of the outer socket 10 and of the inner socket 20 are similar to each other; in particular, the proximal edges 12, 22 have a similar profile and are arranged dorsally and ventrally at different heights, while the medial and lateral regions are curved and formed proximally with respect to the ventral and dorsal portions. Increased lateral stability is thus achieved, at the same time with a high degree of mobility of the intact joint.

FIG. 3 shows a frontal cross-sectional view of an inner socket 20 according to an illustrative embodiment of the prosthesis socket system according to the invention. A peripheral sealing lip 26 formed integrally on the inner socket 20 can be seen here. The sealing lip 26 is arranged in a plane running substantially orthogonally to the longitudinal extent of the inner socket 20 in the proximal-distal direction. In this illustrative embodiment, the flexible inner socket 20 has a material thickness of 3 mm to 4 mm in the medial-lateral direction. The peripheral sealing lip 26 has a material thickness of 0.8 mm in the proximal-distal direction. The peripheral sealing lip 26 is arranged closer to the proximal inner socket opening 24 than to a distal end portion of the inner socket 20. A medial locking nut 28.1 is arranged on the medial face of the inner socket 20. Two lateral locking nuts 28.2 and 28.3 are arranged on the lateral face of the inner socket 20. The number and the arrangement of the locking nuts 28.1, 28.2, 28.3 shown here have been chosen purely by way of example. The locking nuts 28.1, 28.2 and 28.3 serve for screwing to the outer socket 10 (not shown here) of the prosthesis socket system. An inner socket recess 29 is formed in a distal-medial portion of the inner socket 20. The inner socket recess 29 serves to receive, for example, a release valve 30 (not shown here). Alternatively, however, a suction attachment or another means of generating a negative pressure in the inner socket 20 can also be arranged.

FIG. 4 shows a medial cross-sectional view of an inner socket 20 according to another illustrative embodiment of the prosthesis socket system according to the invention. The contour of the inner socket edge 22 runs along a three-dimensional curve from a lower, ventral dorsal portion to an upper, dorsal ventral portion, and, between these, it has a peak in a lateral and/or medial portion. The sealing lip 26 runs substantially completely at a uniform distance from the contour of the inner socket edge 22. In contrast to FIG. 3, the sealing lip 26 is not arranged in one plane but instead runs with the contour of the inner socket edge 22 along a three-dimensional curve. Alternatively, provision is made that the sealing lip 26 runs parallel or approximately parallel to the contour of the inner socket edge 22 or to a contour of the outer socket edge 12 (not shown here) of the outer socket 10 only in parts and, in the remaining regions, has a profile that takes account of the sealing requirements or the comfort needed by the patient.

FIG. 5 shows a frontal view of a prosthesis socket system in the form of a thigh socket, with an outer socket 10 and with an inner socket 20 arranged in the latter.

The screws 18.1, 18.2, 18.3, 18.4 are arranged medially and laterally on the outer socket 10 and fasten the elastic inner socket 20 reversibly to the outer socket 10. A frontal window 16.1, in which an insert 40 is arranged, can be formed or arranged in the outer socket 10, which can be formed as a dimensionally stable component, for example made of a plastic or of a fiber-reinforced composite material. The contour of the insert 40 corresponds substantially to the contour of the window 16.1 but leaves a free space between the outer contour of the insert 40 and the inner contour of the window 16.1. The insert 40 is fastened to the outer socket 10 so as to be adjustable in the window 16.1. The fastening is effected by a clamping element 42, which can be designed as a cable, belt or strap system. The effective length of the clamping element 42 can be increased or shortened via a winding system or a clamping device 31, such that, in the case of a curved contour of the outer socket 10, a radially inwardly directed movement of the insert 40 takes place with shortening of the clamping element 42. If the clamping device 30 is released and the clamping element 42 relaxed, the insert 40 can be moved inside the window 16.1, in particular radially outward, in order to permit an increase in volume inside the inner socket 20 and the outer socket 10. The clamping element 42 is guided in channels 44 inside the outer socket 10 and the insert 40. As an alternative to the clamping element 42 being guided wholly in closed channels 44, the channels can also be partially open, in particular open toward the outside, and can be covered with a filling material or a padding.

An outer socket recess 19, in which the release valve 30 is arranged, is arranged inside the outer socket 10. The clamping device 31, which serves as a winding device for the clamping element 42, is fastened to the outer socket 10 underneath the release valve 30. The clamping device 31 can be rotated in two directions, in order, on the one hand, to wind up the clamping element 42 and, on the other hand, in an opposite direction of rotation, to lengthen the clamping element 42, so that the insert 40 can be loosened. Moreover, the clamping device 31 can be completely decoupled.

FIG. 6 shows a lateral view of the prosthesis socket system with the outer socket 10, and with the inner socket 20 secured reversibly in the latter for the purpose of receiving a thigh stump. In addition to the frontal recess or the frontal window 16.1 and the insert 40 arranged therein, a lateral medial recess 16.2 is formed in the outer socket 10, in which a second insert 40 is mounted. Here too, the outer contour of the second insert 40 is designed corresponding to the inner contour of the second window 16.2, wherein a gap is present between the insert 40 and the edge of the window 16.2. The clamping element 42 is guided in the channels 44 in the insert 40 and the outer socket 10. In the illustrative embodiment shown, the clamping element 42 runs peripherally from the opposite medial lateral side (not shown) over the frontal side to the lateral medial side of the prosthesis socket system, and its two free ends are brought together at the clamping device 31. By rotation in one or the other direction, as indicated by the arrows, the clamping element 42 can be shortened or lengthened by the clamping device 31.

On the rear dorsal side of the outer socket 10, an elastic flexible region 11 is formed which is softer and more flexible and resilient than the rest of the material of the outer socket 10. This makes sitting easier, for example. Additional screws 18.5, 18.6 are arranged in the rear region of the outer socket 10 and connect the inner socket 20 to the outer socket 10.

An attachment device or connector 50, for fastening the prosthesis socket system to a prosthetic knee joint (not shown), is arranged or formed at the distal end of the outer socket 10. The attachment device 50 is designed in particular as an adapter receptacle for a pyramid adapter.

FIG. 7 shows a partial sectional view of the prosthesis socket system with a dimensionally stable, substantially rigid outer socket 10 which is formed at least in some regions on the periphery. The flexible, in particular elastic inner socket 20 is arranged inside the outer socket 10. The inner socket edge 22, which extends radially outward over the outer socket edge 12 in order to cushion the outer socket 10 in the proximal end region, is formed at the proximal end. In the outer socket 10, a window 16.1 is formed in which an insert 40 is arranged. Inside the insert 40, channels 44 are arranged in which the clamping element 42 is guided displaceably. The clamping element 42 is designed for example as a strap, cord, belt or cable; alternative tensioning elements can likewise be used as clamping element 42. On the inner face of the insert 40, i.e. on the face directed toward the inner socket 20, a padding 46 is arranged which protrudes into the interior of the outer socket 10. A continuation of the outer wall of the outer socket 10 would have a substantially rectilinear profile and would bridge the opening 16.1. The padding 46 protrudes radially inward into the interior of the outer socket 10 and, as is shown in FIG. 7, presses the inner socket 20 radially inward in the direction of the thigh stump (not shown). If the clamping element 42 is wound up by the clamping device 31 and its effective length shortened, the clamping element 42 becomes taut between the channel openings in the outer socket 10 and tends to form a straight line. In this way, the insert 40 is moved inward, such that the volume of the inner socket 20 is reduced. In this way, the inner socket 20 is pressed onto the stump (not shown). This can be done, for example, to permit adaptation to a fluctuation in volume of the stump, to a change in the requirements of comfort or to an altered level of activity. For example, if a faster walking speed is sought, the centrifugal forces increase, such that tautness of the clamping element 42 and pressing of the insert 40 with the padding 46 in the direction of the thigh stump are felt to give additional securing.

Protruding radially inward from the inner socket 20 is the sealing lip 26 which, in the illustrative embodiment shown, extends substantially horizontally. In principle, other radially inward profiles of the sealing lip 26 inside the inner socket 20 are also possible. For example, the sealing lip 26 can follow the contour of the outer socket edge 12. It is likewise possible that the sealing lip 26 runs transversely across a window 16.1 and not, as in the illustrative embodiment shown, above the window 16.1. The sealing lip 26 can be arranged all around a window 16.1. The padding 46 can be elastic, and it can likewise be designed as an inflatable cushion of variable volume. The inner contour, that is to say the contour directed toward the inner socket 20, can have a pelotte shape. A shape of the padding 46 adapted to the contour of the body is alternatively provided.

FIG. 8 is a perspective view showing a further variant of the inner socket 20, which is configured with a closed wall and has a proximal edge 22 that is higher at the lateral side in order to permit secure support on the patient over a large surface area. Inside the cavity formed by the inner socket 20, a peripheral seal 26 is integrally formed which protrudes radially inward and, in the fitted state, rests around the stump (not shown). The sealing lip 26 can be designed with a cross section or material thickness that decreases radially from the outside inward, in order to permit particularly flexible support across the whole surface of the patient's stump.

Distally from the sealing lip 26, which is arranged in one plane in the illustrative embodiment shown, knobs 25 or depressions are formed on the inner surface, in order to enlarge the inner surface of the inner socket. Particularly by providing knobs 25 on the inner surface, it is possible to obtain an enlarged support surface and therefore an improved adherence to the stump.

FIG. 9 shows a sectional view of the embodiment of the inner socket 20. In the illustrative embodiment shown, the peripheral sealing lip 26 is formed as a separate, annular sealing lip 26 which is inserted into and secured in a prefabricated groove inside the inner socket 20, for example by adhesive bonding, casting or welding. As an alternative to this, it is possible for the sealing lip 26 to be configured in one piece with the rest of the inner socket 20. Similarly, the knobs 25 can be configured in one piece with the inner socket 20 or can be arranged and fastened on the inner face subsequently, for example by adhesive bonding, casting or welding.

If surface enlargement is to be effected by incorporating depressions in the wall of the inner socket 20, these depressions can be provided during the primary forming process or also at a later stage. In an embodiment with projections on the inner face, these knobs 25 can be arranged so as to extend in peripheral rows substantially parallel to the sealing lip 26. The rows of projections or knobs 25 can be arranged spaced uniformly apart from each other in the proximal-distal direction. It is likewise possible to arrange rows oriented in the proximal-distal direction and alongside one another in the circumferential direction. In addition to the projections being configured solely as knobs, they can also be configured as furrows, grooves or protruding ribs. It is likewise possible for the inwardly protruding projections to be configured differently, for example as ring-shaped, belt-like projections in conjunction with knobs. The material of the projections or knobs 25 can be identical to the material of the rest of the inner socket 20 and can in particular be softer, in order achieve good wearing comfort.

### LIST OF REFERENCE SIGNS

10 outer socket
11 elastic flexible region
12 outer socket edge
14 outer socket opening
16.1 . . . 16.3 windows
18.1 . . . 18.4 screws
19 outer socket recess
20 inner socket
22 inner socket edge
24 inner socket opening
25 knobs
26 sealing lip
28.1 . . . 28.3 locking nuts
29 inner socket recess
30 release valve
31 clamping device
40 insert
42 clamping element
44 channel
46 padding
50 attachment device

1. A prosthesis socket system comprising:
a rigid outer socket, which has a proximal outer socket opening for receiving an extremity stump of a wearer of the prosthesis socket system;
a flexible inner socket, which is connected to the outer socket and has a proximal inner socket opening for receiving the extremity stump;
at least one sealing lip arranged on an inner face of the inner socket directed toward the extremity stump.

2. The prosthesis socket system as claimed in claim 1, wherein the sealing lip is configured as a peripheral sealing lip, which is arranged along a complete inner circumference of the inner face of the inner socket.

3. The prosthesis socket system as claimed in claim 1, further comprising at least one window formed in the outer socket.

4. The prosthesis socket system as claimed in claim 3, wherein the sealing lip is arranged at least partially in a region of the inner socket that is covered by the window.

5. The prosthesis socket system as claimed in claim 3, wherein the outer socket is configured as a lower-leg socket, and the at least one window is arranged in at least one of a frontal shin region or in a region of at least one of a head of a fibula and in a distal calf region.

6. The prosthesis socket system as claimed in claim 3, wherein an adjustable insert, which is fastened to the outer socket, is arranged in the at least one windows.

7. The prosthesis socket system as claimed in claim 1, wherein the sealing lip is arranged closer to the proximal inner socket opening than to a distal end of the inner socket.

8. The prosthesis socket system as claimed in claim 1, wherein the sealing lip is arranged in one plane.

9. The prosthesis socket system as claimed in claim 1, wherein the sealing lip is arranged at least partially parallel to a contour of an inner socket edge surrounding the inner socket opening or to a contour of an outer socket edge surrounding the outer socket opening.

10. The prosthesis socket system as claimed in claim 1, wherein at least one of the sealing lip is cohesively bonded to the inner socket or formed integrally thereon, and the sealing lip and the inner socket are made of the same material.

11. The prosthesis socket system as claimed in claim 1, wherein the prosthesis socket system has at least one of a release valve and a suction attachment for regulating a negative pressure between the extremity stump and the inner socket.

12. The prosthesis socket system as claimed in claim 1, wherein the inner socket and the outer socket are connected to each other releasably and in a manner free of pistoning by a screw connection with locking nuts integrated in the inner socket.

13. The prosthesis socket system as claimed in claim 1, wherein the inner socket is elastic at least in some regions or is made of an elastic material at least in some regions.

14. A prosthesis socket system comprising:
a rigid outer socket having a first inner cavity and a proximal outer socket opening;
a flexible inner socket positioned in the inner cavity and connected to the outer socket, the inner socket comprising:
a second inner cavity to receive a user's residual limb;
a proximal inner socket opening;
an inner face directed toward the residual limb when the residual limb is positioned in the second inner cavity;
at least one sealing lip arranged on the inner face.

15. The prosthesis socket system as claimed in claim 14, wherein the sealing lip is configured as a peripheral sealing lip, which is arranged along a complete inner circumference of the inner face.

16. The prosthesis socket system as claimed in claim 14, further comprising at least one window formed in a sidewall of the outer socket.

17. The prosthesis socket system as claimed in claim 16, wherein the sealing lip is arranged at least partially in a region of the inner socket that overlaps the at least one window.

18. The prosthesis socket system as claimed in claim 16, wherein the outer socket is configured as a lower-leg socket, and the at least one window is arranged in one or more of a frontal shin region, a region of a head of a fibula, and a distal calf region.

19. The prosthesis socket system as claimed in one of claim 16, further comprising an adjustable insert fastened to the outer socket and arranged in the at least one window.

20. The prosthesis socket system as claimed in claim 14, wherein the sealing lip is arranged closer to the proximal inner socket opening than to a distal end of the inner socket.
